(19) United States
(12) Patent Application Publication
(10) Pub. No.: US 2010/0167226 A1
(43) Pub. Date: Jul. 1, 2010

(54) SYSTEM AND METHOD FOR DENTAL APPLICATIONS WITHOUT OPTICAL CONNECTORS IN CONSOLE, AND HANDPIECE ASSEMBLY THEREFOR

(75) Inventors: Gregory B. ALTSHULER, Lincoln, MA (US); Andrei V. BELIKOV, St. Petersburg (RU); Felix I. FELDCHTEIN, Framingham, MA (US)

Correspondence Address: Maria M. Eliseeva, Esq.;Houston Eliseeva LLP, 4 Militia Drive, Ste. 4, Lexington, MA, 02421, US

(73) Assignee: LASER ABRASIVE TECHNOLOGIES, LLC, Quincy, MA (US)

(21) Appl. No.: 12/644,954

(22) Filed: Dec. 22, 2009

Related U.S. Application Data
(63) Continuation of application No. PCT/US2008/068143, filed on Jun. 25, 2008.
(60) Provisional application No. 60/945,994, filed on Jun. 25, 2007.

(51) Int. Cl. A61C 3/00; A61B 6/14

(52) U.S. Cl. 433/29

(57) ABSTRACT

The present invention is directed to systems and methods for dental applications that allow for cost effective, expedient surgical, microsurgical, cosmetic and diagnostics procedures. The system includes a console with no optical connector in it for high optical power and one or more handpiece assemblies detachably connected with the console via electrical connectors. The converter of electrical power to optical power is made as a semiconductor converter, such as a light emitting diode, a diode laser, a diode pumped solid state laser, a diode pumped fiber laser, and the like. The elements of the semiconductor converter in the embodiments of the invention are capable of being positioned in different parts of the handpiece assembly. In one embodiment, the console is adaptive and is capable of executing an executable code stored in a handpiece during its manufacture enabling corresponding performance of the system.

### RELATED APPLICATIONS

This application is a Continuation of International Application No. PCT/US2008/068143 filed on Jun. 25, 2008, which claims the benefit under 35 USC 119(e) of U.S. Provisional Patent Application No. 60/945,994, filed Jun. 25, 2007 entitled, “METHOD AND SYSTEM FOR DENTAL TREATMENT”, both of which are incorporated herein by reference in their entirety.

### FIELD OF THE INVENTION

The subject invention is directed to systems and methods for dental applications that allow for cost effective, expedient dental surgical, microsurgical and diagnostics procedures. In particular, the subject invention is directed to systems and methods for dental applications without optical connectors for high laser radiation in console, a method for adapting the system for a selected dental application and a handpiece assembly for use in the system.

### BACKGROUND OF THE INVENTION

As known in the art, systems for dental applications, such as for various treatment procedures and diagnostic procedures use different types of lasers or non laser light sources for minimum invasiveness, painlessness, and maximum precisions of the procedure. In the dental application field high power dental laser emitting laser energy is used within the oral cavity to treat, evaluate, prevent or diagnose conditions in the oral cavity or the jaw. A high power laser is a laser suitable for drilling, or cutting hard tissue, soft tissue, or dental material, or irreversible modification by phase transition of the structure of hard tissue. A majority of such systems have a laser embedded in the main console. In these systems the system functionality and the range of applications is limited to potentialities of the selected laser. Some other systems have two or more lasers embedded into the console. This approach not only increase flexibility, but also increases system complexity, cost and size as well, the latter being very critical for dental market.

Typically, several types of lasers are used for various dental applications. For example, Erbium (Er) lasers with wavelengths of 2690-2940 nm were proposed and used for hard tissue treatment. CO₂ lasers with wavelengths of 9300-10600 nm and excimer lasers with wavelengths of 194-350 nm are also known to be used for hard tissue treatment. As known in the art, Er lasers and CO₂ lasers are also used for soft tissue treatment, but other lasers with wavelengths of 400-10600 nm produce a better hemostasis effects. In commercial applications, only Er lasers with flashlamp pumping are used for hard tissue treatment. For soft tissue treatment, continuous wave (CW) CO₂ lasers, diode lasers with wavelengths of 800-980 nm or Nd:YAG lasers with a wavelength of 1064 nm are used. Dental practices require treatment of different types of tissues such as enamel, dentine, bone, soft tissues, biofilms, dental materials and other. Different types of tissues, materials and procedures require different light sources. Some manufactures package an Er laser and a soft tissue laser, such as CO₂ in one box. The main disadvantages of this solution are a very high cost and a large size of the device. In addition, this concept has significant cost and size limitation for more than two light sources. Using optical fiber with optical connector on console to deliver high power laser radiation to a distal part of a handpiece in a treatment procedure, is standard for a conventional dental laser system. Such a design is very sensitive to any connector contamination and can be damaged if any dust, particles or absorbing liquid is present on the fiber connector. Another disadvantage of standard optical fiber delivery systems is using a flashlamp pumped Er laser or a CO₂ laser with energy delivery through an IR fiber with low transmission and limited lifetime. Alternative ways, such as delivering energy through an articulated arm or packaging a flashlamp pumped Er laser in a handpiece, are not satisfactory for a dental application, because such a delivery system is extremely bulky. Finally in current laser dentistry for multiple applications it is necessary to equip the dental office with several systems or to use one system with a very limited range of applications. Due to this complexity, the cost of the existing dental lasers is very high and is the main limitation of a widespread use of the laser technology in dentistry.

### SUMMARY OF THE INVENTION

In accordance with the subject invention, there are provided systems and methods for dental applications without optical connectors in console for high optical power, a method for adapting the system for a selected dental application and a handpiece assembly for use in the systems that overcome the above mentioned problems and provide cost effective, expedient dental surgical, microsurgical and diagnostics procedures.

Further, in accordance with the subject invention there is provided a system for dental applications comprising a console and at least one handpiece assembly. The console comprises a console housing, electrical energizing means placed inside the console housing, at least one, but preferably two or more console connector assemblies attached to an exterior of the console housing, and user interface means. Each of the at least one console connector assemblies comprises an electrical unit. The at least one handpiece assembly comprises a proximal part, an umbilical, and a distal part for optical interaction with hard or soft tissue and/or diagnostics or feedback. The proximal part of the at least one handpiece assembly includes a handpiece connector assembly adapted for mating with a corresponding console connector assembly. The proximal part of the at least one handpiece assembly comprises a handpiece electrical connector unit adapted for mating with a corresponding console electrical connector. The umbilical comprises at least one of the group consisting of: optical fiber and electrical wires. The distal part of the at least one handpiece assembly is in communication with the proximal part of the at least one handpiece assembly via the umbilical. The system for dental applications is adapted for performing at least one procedure of the group consisting of: treatment procedures and diagnostic procedures.

In one embodiment of the system for dental applications, the console further comprises hydraulic means placed inside the console housing.

In another embodiment, the console further comprises electronic processing means placed inside the console housing.

In yet another embodiment, the console further comprises pneumatic means placed inside the console housing.

In one embodiment of the system for dental applications, the at least one handpiece assembly is detachably connected with the console via the handpiece connector assembly and corresponding console connector assembly.

In another embodiment, the at least one handpiece assembly further comprises a semiconductor converter of electrical power to optical power.

In one implementation of this embodiment of the system for dental applications, the semiconductor converter of electrical power to optical power is a light emitting diode and is placed in the handpiece connector assembly. In this implementation, the umbilical comprises fiber for delivering light to the distal part of the handpiece assembly.

In another implementation of this embodiment of the system for dental applications, the semiconductor converter of electrical power to optical power is a light emitting diode and is placed in the distal part of the at least one handpiece assembly.

In yet another implementation of this embodiment of the system for dental applications, the semiconductor converter of electrical power to optical power is a diode laser and is placed in the distal part of the at least one handpiece assembly.

In still another implementation of this embodiment of the system for dental applications, the semiconductor converter of electrical power to optical power is a diode pumped solid state laser wherein the diode laser is directly attached and optically coupled with a solid state laser head. In this embodiment, the diode pumped solid state laser is placed in the distal part of the at least one handpiece assembly.

In another embodiment the diode pumped solid state laser may be placed in the connector area and the treatment radiation is delivered by a fiber.

In yet another embodiment the solid state laser can be combined with a nonlinear optical frequency converter, such as harmonic generation, Raman converter, optical parametrical oscillator and others.

In a further implementation of this embodiment of the system for dental applications, the semiconductor converter of electrical power to optical power is a diode pumped solid state laser, wherein the diode laser is placed in the handpiece connector assembly of the at least one handpiece assembly. In this embodiment, the solid state laser is placed in the distal part of the at least one handpiece assembly.

In a still further implementation of this embodiment of the system for dental applications, the semiconductor converter of electrical power to optical power is a diode pumped fiber laser, wherein the diode laser is placed in the handpiece connector assembly. In this implementation, an active fiber is placed in the handpiece connector assembly, or in the umbilical of the at least one handpiece assembly.

In a still further implementation of this embodiment the system for dental applications, the system further comprises a laser unit placed in a distal part of the umbilical, wherein the laser unit is optically coupled with the distal part of the handpiece assembly.

The distal part of the at least one handpiece assembly is preferably comprised in a sheath.

The distal part of the at least one handpiece assembly is preferably detachably connected with the umbilical.

In one embodiment, the system for dental applications further comprises a handpiece kit, the handpiece kit comprising multiple interchangeable shaped head modules and multiple interchangeable tip modules. In one implementation of this embodiment, the distal part of the at least one handpiece assembly is of a modular structure. In this embodiment, the distal part of the at least one handpiece assembly comprises a shaped head module detachably connected with a tip module. The shaped head module and the tip module are selected from the handpiece kit in accordance with a requirement of a treatment procedure or diagnostic procedure.

The at least one handpiece assembly is preferably selected from the group consisting of: a handpiece assembly for laser cutting of soft or hard tissue, a handpiece assembly for dental whitening, a handpiece assembly for optical curing, and a handpiece assembly for optical imaging or diagnostics.

In one embodiment, the at least one handpiece assembly further comprises sensors for feedback control.

In another embodiment, the at least one handpiece assembly further comprises an illumination system.

In yet another embodiment the at least one handpiece assembly further comprises an optical system.

In a further embodiment, the at least one handpiece assembly further comprises recognition means placed in at least one of the head module or the tip module.

In a still further embodiment, the system for dental applications further comprises a system kit. The system kit comprises multiple interchangeable handpiece assemblies, wherein the at least one handpiece assembly is selected from the system kit in accordance with a requirement of a treatment procedure or diagnostic procedure.

In another embodiment of the system for dental applications, the at least one handpiece assembly further comprises handpiece first memory means for storing respective identification data, calibration data, and operational data, and handpiece first output means for outputting the respective identification data calibration data, and operational data. In this embodiment, the console further comprises first receiving means for receiving respective identification data calibration data, and operational data from the at least one handpiece assembly, and console first memory means for storing at least one executable code for a selected dental application, the at least one executable code being downloaded during manufacture of the console. The console also comprises console second memory means for storing corresponding identification data, calibration data, and operational data for the at least one handpiece assembly, and identifying means for identifying the at least one handpiece assembly by associating identification data received from the at least one handpiece assembly with corresponding identification data stored in the console second memory means. Further comprised in the console is retrieving means for retrieving the at least one executable code for a selected dental applications from the console first memory means, setting means for setting calibration and operational parameters of the console in accordance with received calibration and operational data, and management means for executing the at least one executable code downloaded during manufacture of the console. In this embodiment, the user interface means is adapted for displaying at least one handpiece specific user interface in accordance with the at least one executed executable code for a selected dental application downloaded during manufacture of the console.

In yet another embodiment of the system for dental applications, at least one of the at least one handpiece assembly or the console further comprises additional memory means for storing a compatibility matrix and compatibility checking means for checking compatibility of the at least one handpiece assembly with the console by associating software and hardware versions of the handpiece assembly with software and hardware versions of the console. In this embodiment, the compatibility matrix is capable of being multidimensional.

In a further embodiment of the system for dental applications the console is made adaptive. In this embodiment the at least one handpiece assembly further comprises handpiece first memory means for storing respective identification data, calibration data, and operational data, and handpiece first output means for outputting the respective identification data calibration data, and operational data. Also comprised in the at least one handpiece assembly is handpiece second memory means for storing at least one first executable code for a selected dental application associated with the adaptive console, and handpiece second output means for outputting the at least one first executable code for a selected dental applications associated with the adaptive console. In this embodiment the adaptive console further comprises first receiving means for receiving respective identification data calibration data, and operational data from the at least one handpiece assembly, and second receiving means for receiving from the at least one handpiece assembly the at least one first executable code for a selected dental application associated with the adaptive console. Further comprised in the adaptive console is adaptive console first memory means for storing corresponding identification data, calibration data, and operational data for the at least one handpiece assembly, and adaptive console second memory means for storing the at least one first executable code for a selected dental application associated with the adaptive console received from the at least one handpiece assembly. Yet further, the adaptive console comprises identifying means for identifying the at least one handpiece assembly by associating identification data received from the at least one handpiece assembly with corresponding identification data stored in the adaptive console first memory means, and setting means for setting calibration and operational parameters of the adaptive console in accordance with received calibration and operational data. Still further, in this embodiment, the adaptive console comprises first retrieving means for retrieving from the adaptive console second memory means the at least one first executable code received from the at least one handpiece assembly, and first management means for executing the at least one first executable code for a selected dental application received from the at least one handpiece assembly. In this embodiment, the user interface means is adapted for displaying at least one first handpiece specific user interface in accordance with the at least one first executed executable code for a selected dental application received from the at least one handpiece assembly.

In another embodiment of the system for dental applications, the adaptive console further comprises adaptive console third memory means for storing at least one second executable code for a selected dental application downloaded during manufacture of the adaptive console, second retrieving means for retrieving the at least one second executable code for a selected dental application downloaded during manufacture of the adaptive console, and second management means for executing the at least one second executable code for a selected dental application retrieved by the second retrieving means. In this embodiment, the user interface means is further adapted for displaying at least one second handpiece specific user interface in accordance with the at least one second executed executable code for a selected dental application downloaded during manufacture of the adaptive console.

In yet another embodiment of the system for dental applications, at least one of the at least one handpiece assembly or the adaptive console further comprises additional memory means for storing a compatibility matrix and compatibility checking means for checking compatibility of the at least one handpiece assembly with the adaptive console by associating software and hardware versions of the handpiece assembly with software and hardware versions of the adaptive console.

In one embodiment of the system for dental applications, the compatibility matrix is multidimensional.

In one embodiment of the system for dental applications, if multiple first executable codes are stored in handpiece second memory means, the multiple first executable codes belong to different platforms.

The user interface means preferably includes a graphical display.

In one embodiment of the system for dental applications, the at least one procedure is selected from procedures for oral cavity treatment, evaluation, prevention or diagnose conditions in the oral cavity or the jaw with light sources suitable for drilling, or cutting hard tissue, soft tissue, or dental material, or irreversible modification by phase transition of the structure of hard tissue.

In another embodiment of the system for dental applications, the at least one procedure is selected from the group of diagnostic procedures consisting of: optical imaging, translucent imaging, fluorescent imaging, fluorescent spectroscopy, fluorescent imaging, confocal microscopy, multiphoton microscopy, reflectometry and optical coherence tomography.

Further, in accordance with the subject invention, there is provided a system for dental applications comprising a console and at least one handpiece assembly for optical interaction with hard or soft tissue. The console comprises a console housing, electrical energizing means placed inside the console housing, and at least one console connector assembly attached to an exterior of the console housing. The at least one handpiece assembly comprises a handpiece connector assembly adapted for mating with a corresponding console connector assembly, handpiece first memory means for storing respective identification data, calibration data, and operational data, and handpiece first output means for outputting the respective identification data calibration data, and operational data. In this system, the console further comprises first receiving means for receiving respective identification data calibration data, and operational data from the at least one handpiece assembly, and console first memory means for storing at least one executable code for a selected dental application, the at least one executable code being downloaded during manufacture of the console. Further comprised in the console is console second memory means for storing corresponding identification data, calibration data, and operational data for the at least one handpiece assembly, and identifying means for identifying the at least one handpiece assembly by associating identification data received from the at least one handpiece assembly with corresponding identification data stored in the console second memory means. Still further, the console comprises retrieving means for retrieving the at least one executable code for a selected dental applications from the console first memory means, and setting means for setting calibration and operational parameters of the console in accordance with received calibration and operational data. In addition, the console comprises management means for executing the at least one executable code downloaded during manufacture of the console, and user interface means adapted for displaying at least one handpiece specific user interface in accordance with the at least one executed executable code for a selected dental application downloaded during manufacture of the console. The system for dental applications is adapted for performing at least one procedure of the group consisting of: treatment procedures and diagnostic procedures.

In one embodiment, at least one of the at least one handpiece assembly or the console further comprises additional memory means for storing a compatibility matrix and compatibility checking means for checking compatibility of the at least one handpiece assembly with the console by associating software and hardware versions of the handpiece assembly with software and hardware versions of the console. The compatibility matrix is capable of being multidimensional.

Further, in accordance with the subject invention, there is provided a system for dental applications comprising an adaptive console and at least one handpiece assembly for optical interaction with hard or soft tissue. The console comprises a console housing, electrical energizing means placed inside the console housing, and at least one console connector assembly attached to an exterior of the console housing. The at least one handpiece assembly comprises a handpiece connector assembly adapted for mating with a corresponding console connector assembly, handpiece first memory means for storing respective identification data, calibration data, and operational data, and handpiece first output means for outputting the respective identification data calibration data, and operational data. Further comprised in the at least one handpiece assembly is handpiece second memory means for storing at least one first executable code for a selected dental application associated with the adaptive console, and handpiece second output means for outputting the at least one first executable code for a selected dental applications associated with the adaptive console. In this system, the adaptive console further comprises first receiving means for receiving respective identification data calibration data, and operational data from the at least one handpiece assembly, and second receiving means for receiving from the at least one handpiece assembly the at least one first executable code for a selected dental application associated with the adaptive console. Also comprised in the adaptive console is adaptive console first memory means for storing corresponding identification data, calibration data, and operational data for the at least one handpiece assembly, and adaptive console second memory means for storing the at least one first executable code for a selected dental application associated with the adaptive console received from the at least one handpiece assembly. The adaptive console further comprises identifying means for identifying the at least one handpiece assembly by associating identification data received from the at least one handpiece assembly with corresponding identification data stored in the adaptive console first memory means, and setting means for setting calibration and operational parameters of the adaptive console in accordance with received calibration and operational data. In addition, the adaptive console comprises first retrieving means for retrieving from the adaptive console second memory means the at least one first executable code received from the at least one handpiece assembly, first management means for executing the at least one first executable code for a selected dental application received from the at least one handpiece assembly, and user interface means adapted for displaying at least one first handpiece specific user interface in accordance with the at least one first executed executable code for a selected dental application received from the at least one handpiece assembly. The system for dental applications is adapted for performing at least one procedure of the group consisting of: treatment procedures and diagnostic procedures.

In one embodiment of this system, at least one of the at least one handpiece assembly or the console further comprises additional memory means for storing a compatibility matrix and compatibility checking means for checking compatibility of the at least one handpiece assembly with the console by associating software and hardware versions of the handpiece assembly with software and hardware versions of the console. The compatibility matrix is capable of being multidimensional.

Still further, in accordance with the subject invention, there is provided a method for adapting a system for selected dental applications in accordance with the system as set forth above.

Yet further, in accordance with the subject invention, there is provided a handpiece assembly for dental applications comprising a proximal part including a handpiece connector assembly adapted for mating with a corresponding console connector assembly of an associated console, an umbilical comprising at least one of the group consisting of: optical fiber and electrical wires, and a distal part for optical interaction with hard or soft tissue. The distal part of the handpiece assembly is in communication with the proximal part of the handpiece assembly via the umbilical. The handpiece assembly is adapted for performing at least one procedure of the group consisting of: treatment procedures and diagnostic procedures.

In one embodiment, the handpiece assembly is capable of being detachably connected with an associated console via the handpiece connector assembly and corresponding associated console connector assembly.

In another embodiment, the handpiece assembly further comprises a semiconductor converter of electrical power to optical power.

In one implementation of this embodiment of the handpiece assembly, the semiconductor converter of electrical power to optical power is a light emitting diode and is placed in the handpiece connector assembly. In this implementation, the umbilical comprises fiber for delivering light to the distal part of the handpiece assembly.

In another implementation of this embodiment, of the handpiece assembly the semiconductor converter of electrical power to optical power is a light emitting diode and is placed in the distal part of the at least one handpiece assembly.

In yet another implementation of this embodiment of the handpiece assembly, the semiconductor converter of electrical power to optical power is a diode laser and is placed in the distal part of the at least one handpiece assembly.

In still another implementation of this embodiment of the handpiece assembly, the semiconductor converter of electrical power to optical power is a diode pumped solid state laser wherein the diode laser is directly attached and optically coupled with a solid state laser head. In this embodiment, the diode pumped solid state laser is placed in the distal part of the at least one handpiece assembly.

In a further implementation of this embodiment of the handpiece assembly, the semiconductor converter of electrical power to optical power is a diode pumped solid state laser, wherein the diode laser is placed in the handpiece connector assembly of the at least one handpiece assembly. In this embodiment, the solid state laser is placed in the distal part of the at least one handpiece assembly.

In a still further implementation of this embodiment of the handpiece assembly, the semiconductor converter of electrical power to optical power is a diode pumped fiber laser, wherein the diode laser is placed in the handpiece connector assembly. In this implementation, an active fiber is placed in the handpiece connector assembly, or in the umbilical of the at least one handpiece assembly.

In a still further implementation of this embodiment the handpiece assembly further comprises a laser unit placed in a distal part of the umbilical, wherein the laser unit is optically coupled with the distal part of the handpiece assembly.

In a still further implementation of this embodiment of the handpiece assembly, the semiconductor converter of electrical power to optical power is a diode pumped solid state laser, wherein the diode laser is directly attached and optically coupled with a first solid state laser head and placed in the handpiece connector assembl. A second solid state laser head is placed in the distal part of the handpiece assembly, wherein the umbilical comprises fiber for delivering light to the a second solid state laser head.

In a further embodiment of the handpiece assembly, the distal part of the handpiece assembly is comprised in a sheath.

In a still further embodiment of the handpiece assembly, the distal part of the handpiece assembly is detachably connected with the umbilical.

In a yet further embodiment of the handpiece assembly, the latter further comprises a handpiece kit, the handpiece kit comprising multiple interchangeable shaped head modules and multiple interchangeable tip modules.

In another embodiment of the handpiece assembly, the distal part of the handpiece assembly is of a modular structure, wherein the distal part of the handpiece assembly comprises a shaped head module detachably connected with a tip module. In this embodiment, the shaped head module and the tip module are selected from the handpiece kit in accordance with a requirement of a treatment procedure or diagnostic procedure.

In a further embodiment, the handpiece assembly is selected from a handpiece assembly for oral cavity treatment, evaluation, preventing or diagnose conditions in the oral cavity or the jaw with light sources suitable for drilling, or cutting hard tissue, soft tissue, or dental material, or irreversible modification by phase transition of the structure of hard tissue.

In a still further embodiment, the handpiece assembly further comprises sensors for feedback control.

The handpiece assembly preferably comprises an illumination system.

In one embodiment, the handpiece assembly further comprises an optical system.

The embodiments of the subject invention presented herein expand the class of systems for dental applications, being designed both for treatment and diagnostic procedures. The main console has no optical connectors in it, allowing for cost effective, expedient dental, surgical, microsurgical and diagnostics procedures. Specific types and implementations of the system as well as of its elements, in particular, the handpiece assembly, the implementations and locations of the semiconductor converter and other elements, advantageously define the invention in its particular embodiments. The converter of electrical power to optical power is made as a semiconductor converter, such as a light emitting diode, a diode laser, a diode pumped solid state laser, a diode pumped fiber laser, and the like. The elements of the semiconductor converter in the embodiments of the invention are capable of being positioned in different parts of the handpiece. In one embodiment, the console is adaptive and is capable of executing an executable code stored in a handpiece during its manufacture enabling corresponding performance of the system. In another embodiment, at least one of the console or the handpiece assembly stores a compatibility matrix, whereupon a compatibility check is performed either by the console or the handpiece assembly by associating software and hardware versions of the handpiece assembly with software and hardware versions of the console. The subject method for adapting the main console disclosed herein provides effective performance of the system in various dental applications.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a schematic diagram of a system for dental applications according to one embodiment of the subject invention;

FIG. 2 is a schematic diagram of a system for dental applications according to another embodiment of the subject invention;

FIG. 3 illustrates an implementation of a system for dental applications according to another embodiment of the subject invention;

FIG. 4 is a schematic diagram of a system for dental applications according to another embodiment of the subject invention; and

FIG. 5 is a schematic diagram of a system for dental applications according to another embodiment of the subject invention;

FIG. 6 is a schematic diagram of a system for dental applications according to another embodiment of the subject invention;

FIG. 7 is a schematic diagram of a system for dental applications according to another embodiment of the subject invention;

FIG. 8 is flowchart illustrating a method for adapting a system for selected dental applications according to one embodiment of the present invention from the handpiece side;

FIG. 9 is a flowchart illustrating a method for adapting a system for selected dental applications according to one embodiment of the present invention from the console side;

FIG. 10 is a flowchart illustrating a method for adapting a system for selected dental applications according to another embodiment of the present invention from the handpiece side; and

FIG. 11 is a flowchart illustrating a method for adapting a system for selected dental applications according to another embodiment of the present invention from the console side.

### DETAILED DESCRIPTION OF THE PREFERRED EMBODIMENTS

The subject invention is directed to systems and methods for dental applications without optical connectors for high laser radiation in console, a method for adapting the system for a selected dental application and a handpiece assembly for use in the systems that overcome the above mentioned problems and provide cost effective, expedient dental surgical, microsurgical and diagnostics procedures. In the dental application field high power dental laser emitting laser energy is used within the oral cavity to treat, evaluate, prevent or diagnose conditions in the oral cavity or the jaw. A high power laser is a laser suitable for drilling, or cutting hard tissue, soft tissue, or dental material, or irreversible modification by phase transition of the structure of hard tissue. Dental material includes any artificial material used in oral cavity and jaw like dental filler, veneer, crown, implant, bone implant coating of hard tissue, and bracket.

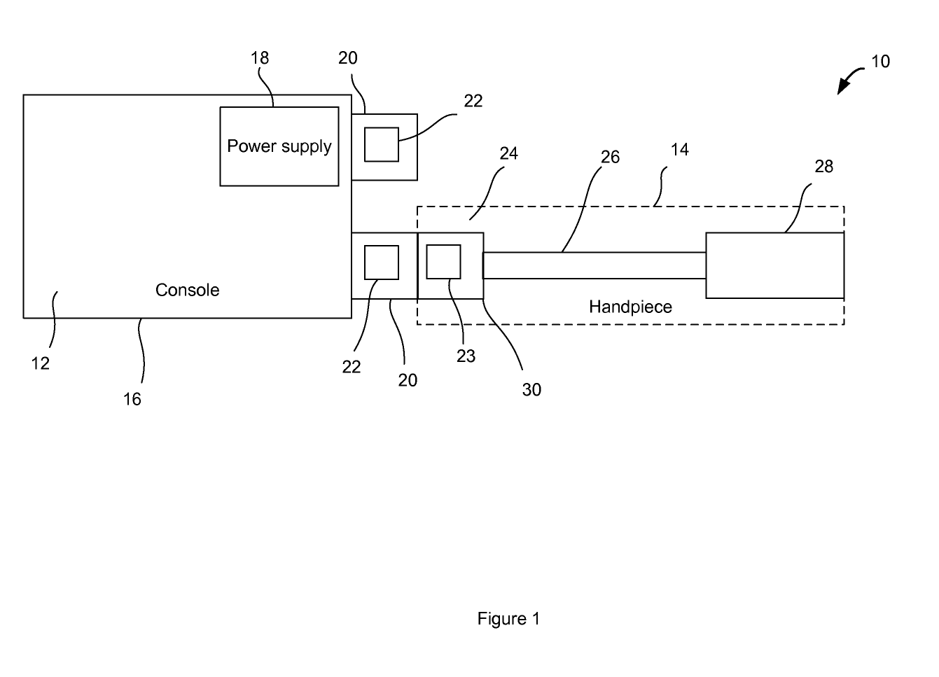

Referring now to FIG. 1, there is shown a schematic block diagram of a system 10 for dental applications in accordance with one embodiment of the subject invention. As shown in FIG. 1, the system 10 includes a console 12 and a handpiece assembly 14. The console 12 comprises a console housing 16, a power supply 18 placed inside the console housing 16, two console connector assemblies 20, attached to an exterior of the console housing 16, and user interface means not shown in FIG. 1. Shown in FIG. 1 is a system 10, in which the console 12 includes two console connector assemblies 20. It will be appreciated, however, that the system 10 is capable of including one or multiple console connector assemblies 20 without departing from the scope of the subject invention. Each of the console connector assemblies 20 comprises a respective console electrical connector unit 22. The connector assembly 20, in addition to the console electrical connector unit 22 with signal and high power contacts, may further include additional connectors to deliver water, air or other liquids and gas to cool optical sources and to irrigate treated tissues, as well as to energize scanners or other periphery devices in the handpiece assembly. The connector assembly 20 may also include low-power optical connectors used to deliver low power light for communication, feedback or diagnostic purposes (these elements not shown in the drawing). The handpiece assembly 14 comprises a proximal part 24, an umbilical 26, and a distal part 28 for optical interaction with hard or soft tissue or other subjects in oral cavity, such as bacteria, biofilm, or dental material.

The proximal part 24 of the handpiece assembly 14 includes a handpiece connector assembly 30 adapted for mating with a corresponding console connector assembly 20, wherein the handpiece connector assembly 30 includes a handpiece electrical connector unit 23 adapted for mating with a corresponding console electrical connector unit 22. It will be appreciated that handpiece connector assembly 30 may include additional connectors, such as for water, air, or gas delivery, or other suitable connectors, as known in the art, the latter being adapted for mating with respective connectors in the console connector assemblies 20 (additional connectors not shown). The umbilical 26 comprises at least one of the group consisting of: optical fiber and electrical wires (not shown in the drawing). The distal part 28 of the handpiece assembly 14 is in communication with the proximal part 24 of the handpiece assembly 14 via the umbilical 26. The system 10 for dental applications is adapted for performing at least one procedure of the group consisting of: treatment procedures and diagnostic procedures.

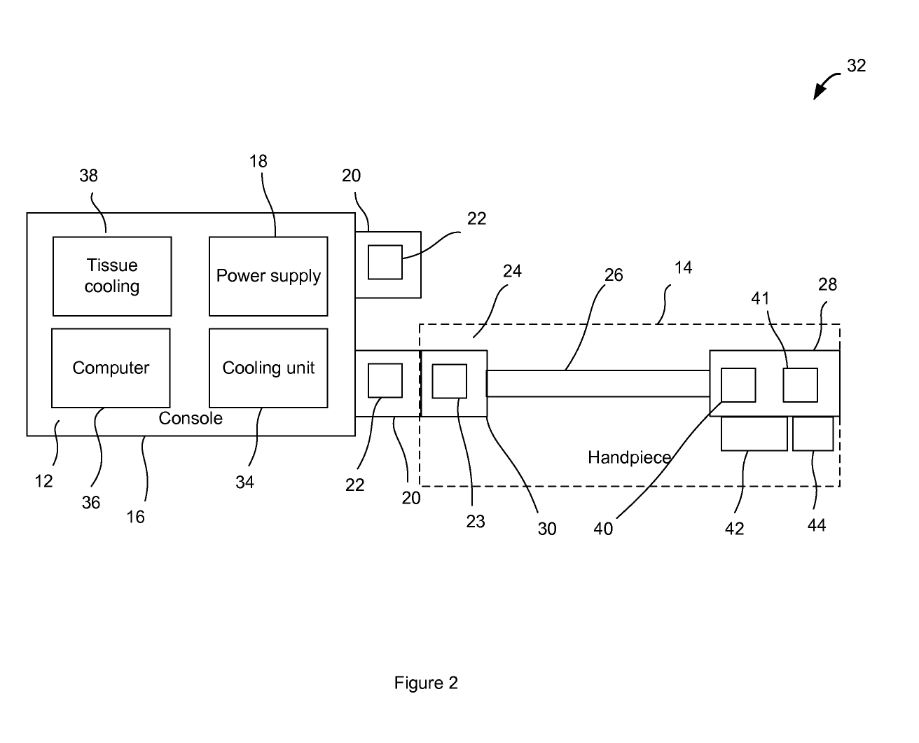

Turning now to FIG. 2, there is shown a schematic block diagram of a system 32 for dental applications in accordance with another embodiment of the subject invention. As shown in FIG. 2, in the system 32 the console 12, in addition to the elements indicated in FIG. 1, further comprises a cooling unit 34, a computer unit 36, and a tissue cooling unit 38 placed inside the console housing 16. The system 32 is also includes a tissue cooling unit placed inside the console housing 16. In the embodiment of FIG. 2, the handpiece assembly 14 comprises a semiconductor converter of electrical power to optical power, which is a light emitting diode 40 and is placed in the distal part 28 of the handpiece assembly 14. Also included in the handpiece assembly 14 of the embodiment shown in FIG. 2, is an optical system 41, a tissue cooling unit 42, and a tip 44. It will be understood that the cooling unit 34 is capable of being used for cooling the light emitting diode 40 and potentially other components inside and outside the console housing 16.

In another embodiment, the semiconductor converter of electrical power to optical power is capable of being implemented as a diode laser, which is placed in the distal part 28 of the handpiece assembly 14, analogous to that of the light emitting diode 38 in the embodiment of FIG. 2 (this embodiment is not shown in the drawings). In yet another embodiment, the semiconductor converter of electrical power to optical power is a diode pumped solid state laser, wherein the diode laser is directly attached and optically coupled with a solid state laser head, and wherein the diode pumped solid state laser is placed in the distal part 28 of the handpiece assembly 14, analogous to that of the light emitting diode 38 in the embodiment of FIG. 2 (this embodiment is not shown in the drawings). Those skilled in the art will recognize that the solid state laser is capable of being implemented, for example, as an active ions doped crystal, glass or fiber.

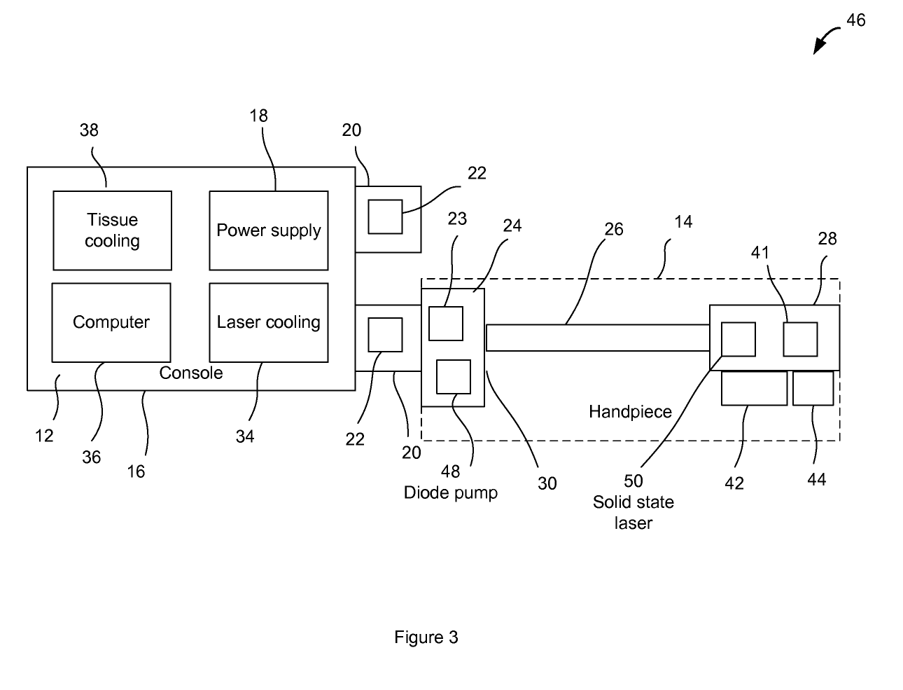

Referring now to FIG. 3, there is shown a schematic block diagram of a system 46 for dental applications in accordance with another embodiment of the subject invention. In the system 46 the semiconductor converter of electrical power to optical power is a diode pumped solid state laser. In the embodiment of FIG. 3, the diode laser 48 is placed in the handpiece connector assembly 30 of the handpiece assembly 14. The solid state laser 50 is placed in the distal part 28 of the handpiece assembly 14. To minimize the size of the distal part 28, the solid state laser 50 is capable of being placed in the umbilical 26. In this case pumping power from the diode laser 48 is delivered to the solid state laser 50 through an optical fiber and power from the solid-state laser 50 is delivered to the distal part 28 through another optical fiber. Silica optical fiber may be used for pumping power delivery from the diode laser to the solid state laser. Silica optical fiber has good transmission for wavelengths from 300 to approximately 2300 nm, where most absorption band spectrum of solid state laser materials is located. If the solid state laser has an emitting wavelength longer than 2300 nm, then the preferable location of the solid state laser 50 is the distal part 14. In a case when the solid state laser 50 is located in the umbilical 26, then infrared fiber such as sapphire or Ge oxide glass can be used for delivery of solid state laser emission to the optics 41. The length of infrared fiber can be very short (1 to 10 cm) to prevent losses of laser energy.

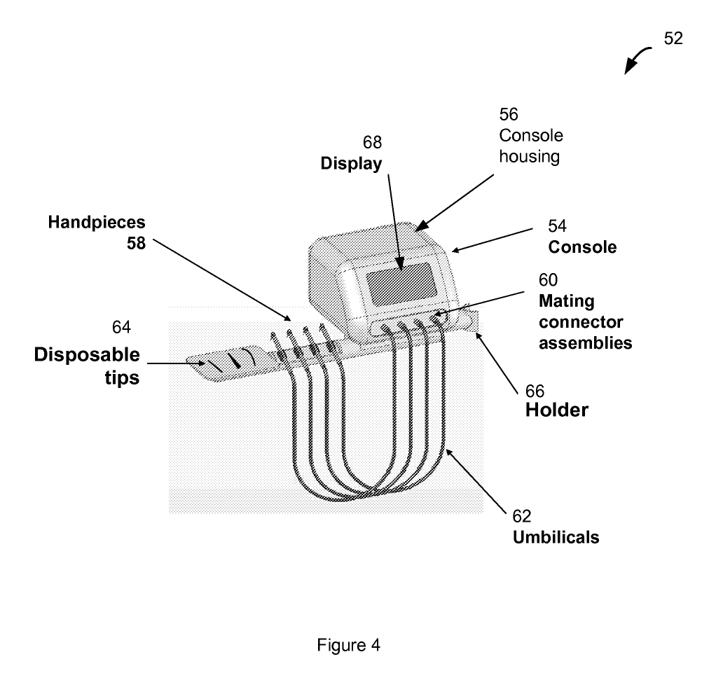

FIG. 4 illustrates one embodiment of a system 52 for dental applications in accordance the subject invention. Shown in FIG. 4 is a console 54 with a console housing 56 and four handpieces 58 attached to the console 54 via respective mating console connector assemblies, attached to an exterior of the console housing 56, and handpiece connector assemblies 60. Also shown in FIG. 4 are umbilicals 62, disposable tips 64 and a holder 66 for placing the system 52 for user convenience. The console 54 of the system 52 also includes a display 68. Those skilled in the art will appreciate that four handpieces 58 attached to the console 54 are shown in FIG. 4 for example purpose only.

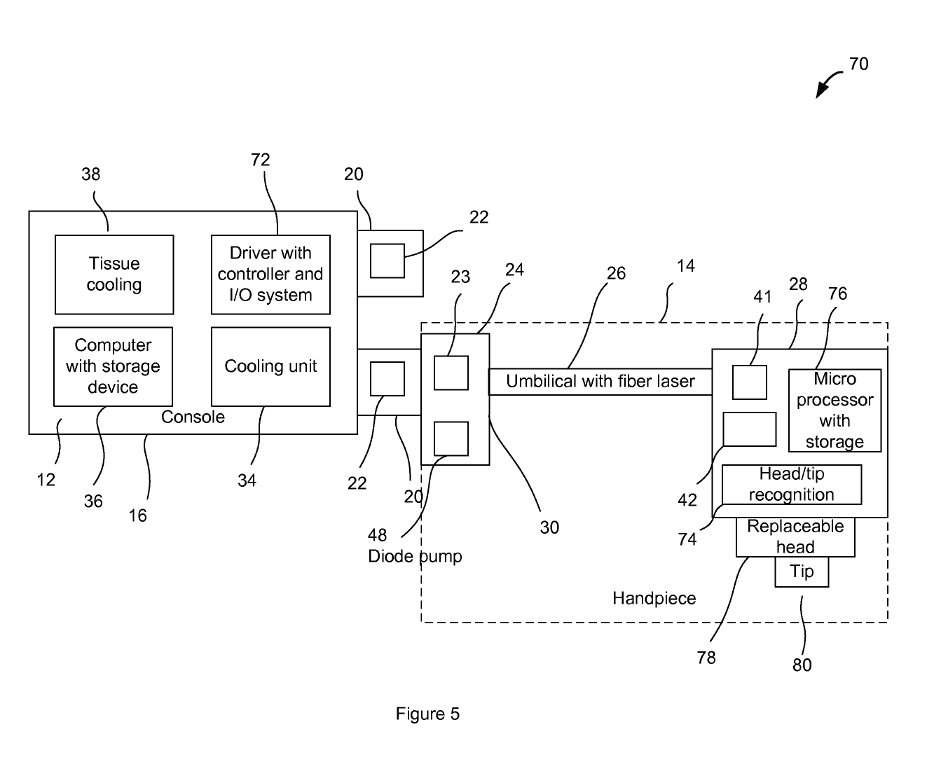

Turning now to FIG. 5, there is shown a schematic block diagram of a system 70 for dental applications in accordance with another embodiment of the subject invention. In the system 70, the console 12 includes a power supply 18. The power supply 18, as known in the art, is capable of including several components to convert electrical power from the AC wall plug to form an electrically energized optical source. A typical power supply system includes an AC to DC converter, a driver forming pump voltage or current in the form of constant current, modulated current or pulsed current. Modulated or pulsed current may have different temporal structure and formed with analog or digital means. The driver operation is typically controlled with a controller containing an embedded microprocessor or digital signal processor (DSP). The controller further includes an input/output system, which can monitor laser parameters, treatment parameters and environment parameters, so the controller is capable of modifying or terminating system operation based on the input sensors, such as temperature sensors, pressure sensors, optical sensors, electrical sensors, magnetic sensors, acoustic sensors, fluorescence sensors, interlock sensors and the like. The controller is also capable of controlling system operation by using an output system controlling laser shutter, beam scanners, power attenuation, tissue irrigation and other functions. In this embodiment the semiconductor converter of electrical power to optical power is a diode pumped fiber laser. The diode laser 48 is placed in the handpiece connector assembly 30, wherein an active fiber is placed in the umbilical 26 of the handpiece assembly 14 (the active fiber not shown in the drawing). The active fiber is also capable of being placed in the handpiece connector assembly 30 (this embodiment is not shown in the drawing). In the system 70 the handpiece 14 further includes a head and tip recognition unit 74, a microprocessor 76 with a data storage device, a replaceable head 78, and replaceable tip or spacer/explorer 80. The microprocessor 76 with a data storage device is placed in the handpiece connector assembly 30. The replaceable head 78 can include different optical systems to adjust beam size for different tips and procedures. The replaceable tip 80 may be optimized for different procedures by different shape, size and/or material. For example, side firing tip with a small diameter as 200-600 micrometers can be used for endodontic treatment, sapphire knife type tip can used for gingival contouring using opto-mechanical cutting.

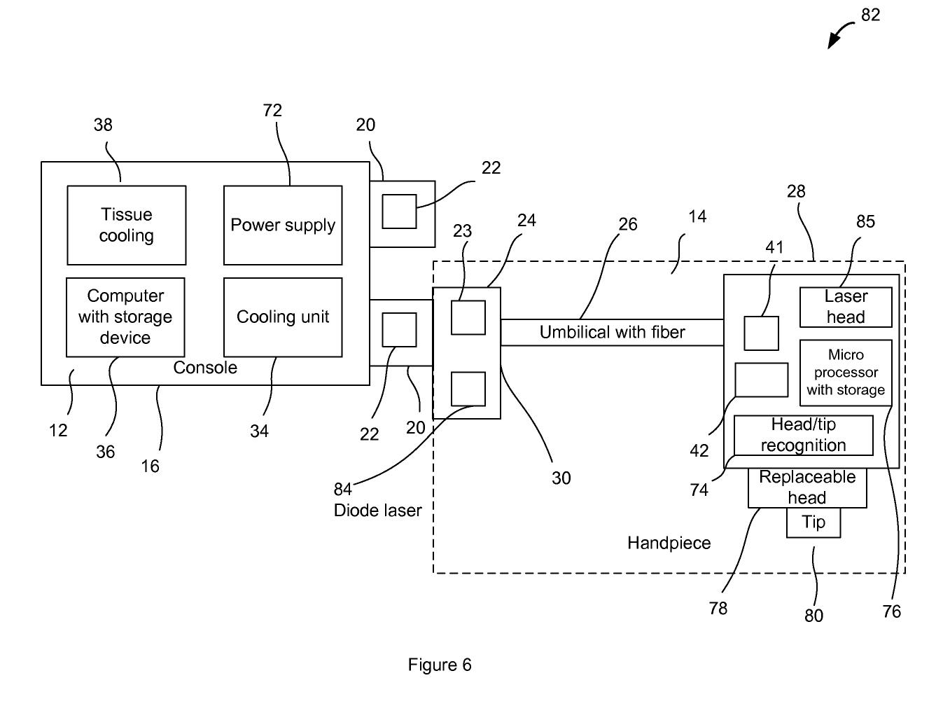

Referring now to FIG. 6, there is shown a schematic block diagram of a system 82 for dental applications in accordance with another embodiment of the subject invention. In the system 82, the semiconductor converter of electrical power to optical power includes a diode laser 84 placed in the handpiece connector assembly 30, and a solid state laser head 85 placed in the distal part 28 of the handpiece 14. The diode laser 84 in this embodiment is capable of emitting wavelengths in the range 800-2600 nm which have good transmission through silica fiber. The solid state laser head 85 may comprise an Er or Ho crystal laser with emission in the range 1500-1800 nm, 1850-2100 nm and 2650-3000 nm, Er glass laser with a wavelength of about 1540 nm, Cr:ZnSe laser with tunable wavelengths in the range of 1500-2800 nm. The diode laser 84 may be comprised of individual emitters, bars and stack of bars coupled in one or several optical fibers using different optical systems such as microlenses, lenses and fiberoptic beam combiners.

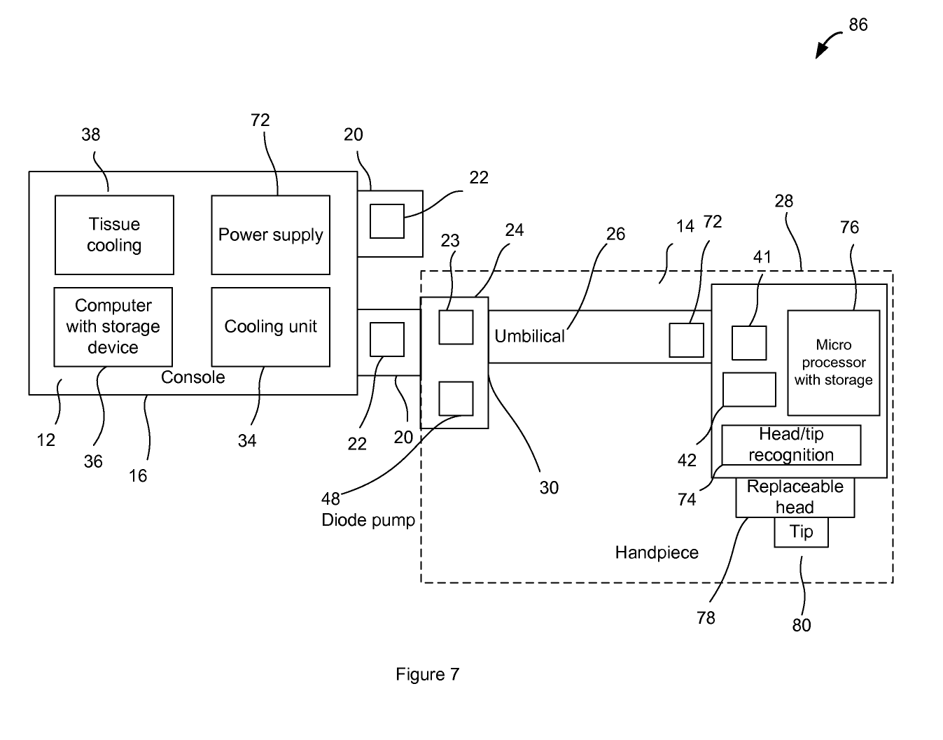

Turning now to FIG. 7, there is shown a schematic block diagram of a system 86 for dental applications in accordance with another embodiment of the subject invention. The system 86 further comprises a laser unit 72 placed in a distal part of the umbilical 26 to minimize size of handheld distal part 14, wherein the laser unit 72 is optically coupled with the distal part 28 of the handpiece assembly 14. In this embodiment, the laser unit 72 is capable of being implemented as solid state laser. The optical coupling is capable of being provided with the use of a mirror, or an articulated arm with mirrors, or with a short fiber, as known in the art. It will be understood that in accordance with this embodiment, multiple implementations are capable of being provided. For example, in one implementation, the semiconductor converter of electrical power to optical power includes a diode laser placed in the laser unit 72, and a solid state laser head placed in the distal part 28 of the handpiece 14 (not shown in the drawing). In another implementation, a diode laser is placed in the unit 72, and a solid state laser is in the distal part 28 of the handpiece assembly 14 (not shown in the drawing). In this embodiment, pumping power from the diode laser 48 is delivered to the solid state laser 72 through an optical fiber and the power from the solid-state laser is delivered to the distal part 28 through another optical fiber or mirrors or other waveguide means. Silica optical fiber can be used for delivering pumping power from the diode laser 48 to the solid state laser 72. Silica optical fiber has good transmission for wavelength range from 300 to 2300 nm, where the most absorption bands of the solid state laser materials are located. If the solid state laser 72 has an emitting wavelength longer than 2300 nm then the preferable location of the solid state laser 72 is the distal part 28 of the handpiece 14. When the solid state laser 72 with such a wavelength is located in the umbilical 26, infrared fiber, such as sapphire or Ge oxide glass can be used for delivery of solid state laser emission to the optics 41. The length of the infrared fiber in this case can be very short (1 to 10 cm) to prevent losses of laser energy.

In this example, one handpiece assembly 14 can be a handpiece for hard tissue drilling and soft tissue minimum invasive cutting, which includes a diode pumped Er laser with wavelengths 2700-3000 nm, energy per pulse 1-1000 mJ, average power 1-10 W and pulsewidth 1-1000 microsecond. This handpiece may be equipped with different replaceable heads for hard and soft tissue microperforation, fast drilling, periodontal and endodontic tips and others. Another handpiece assembly 14 can be a Er laser handpiece for laser texturing of hard tissue surface or dental material for enhanced bonding and sintering nano and micro particles in hard tissue or dental material surface. This laser has similar parameters but includes a scanner in the distal part 28 of the handpiece assembly 14. A third handpiece assembly 14 can be a handpiece for soft tissue treatment with a large coagulation zone for better homeostasis with a diode laser generating wavelength in the range 500-1350 nm and power 1-100 W. A fourth handpiece assembly 14 can be a handpiece for soft tissue treatment with minimum necrotic zone for minimum healing time and precise tissue cutting with a diode laser generating wavelength of 300-450 nm or 1350-3000 nm and power 0.1-100 W. A multi wavelength diode laser is also capable of being used that generates different combinations of wavelengths from 410 nm, 577 nm, 975 nm, 1470 nm, 1890 nm and 2940 nm. These wavelengths match different peaks of blood and water absorption, the latter being the main chromophore of oral soft tissue. Combining these wavelengths allows for a precisely optimized therapeutic effect for different types of tissue. Fewer or more other handpiece assemblies 14 are capable of being attached to the console 12 depending on the system design and user necessities to optimize the treatment or diagnostics procedure. For example, in addition to the four described above handpiece assemblies 14, several other handpiece assemblies 14 may be attached to the same console 12. For example, a handpiece assembly 14 with high power LED for curing with power 1-20 W, a handpiece assembly 14 with high power LED or laser for tooth whitening with power 1-100 W, a handpiece assembly 14 for bacterial reduction and biofilm treatment with LED or laser wavelength 300-1000 nm and power 1-100 W, a handpiece assembly 14 for dental tissue healing and regeneration including bone tissue with LED or a laser with wavelength of 600-1350 nm and power 1-100 W. An additional handpiece can be specialized for periodontal treatment including root scaling, de epitalization and initiation of reattachment hard and soft tissue. This handpiece is capable of including ultrasound sources for enhanced scaling by combining mechanical and light energy. An additional handpiece can be specialized for root canal treatment including microperforation and bacteria sterilization. An additional handpiece can be specialized for implatology including precise angle controlled bone perforation.

In one embodiment, the semiconductor converter of electrical power to optical power is a diode pumped solid state laser, wherein the diode laser is directly attached and optically coupled with a first solid state laser head and placed in the handpiece connector assembly. In this embodiment (not shown in the drawings), a second solid state laser head is placed in the distal part of the handpiece assembly, wherein the umbilical comprises fiber for delivering light to the a second solid state laser head.

In addition, a handpiece assembly 14 for optical dental diagnostics can be attached to the same platform. It may be an optical reflectometer for caries detection using tooth color diagnostics, fluorescence detector of biofilm presence and caries diagnostic, optical dental camera, optical coherent tomography imager and other. All semiconductor light sources such as diode lasers or LED in multiple application handpiece assemblies 14 can be supported from the same console 12 with a universal power supply, computer and cooling system. Connector assemblies 20, 22 do not include optical connectors for high power optical radiation and can be easy and reliably disconnected and reconnected multiple times including changing during the treatment of one patient.

The dental system proposed in present invention includes a console, which can support all described handpieces and specialized applications. Each handpiece may be improved by using different replicable heads and tips and operation software. The dental system described herein is adapted for maximum applications using optical energy with minimum cost.

The described dental light based system can be used for treatment of different conditions including, but not limited to caries therapy, crown preparation, hard tissue microtexturing for better bonding, hard tissue modification for caries prevention, hypersensitivity reduction, and tooth whitening, bone and soft tissue cutting for implantology, periodontal and endodontic treatment, gingival and other soft tissue cutting, gingival troughing, enameloplasty, excavation of pits and fissures for placement of sealants, root canal preparation including enlargement, root canal debridement and cleaning, cutting, shaving, contouring and resection of oral osseous tissues (bone), osteotomy, apicoectomy—amputation of the root end, periodontal procedures including, full, partial and split thickness flap, laser removal of diseased, infected, inflamed, and necrosed soft tissue within the periodontal pocket, removal of highly inflamed edematous tissue affected by bacteria penetration of the pocket lining and junctional epithelium, removal of granulation tissue from bony defects, sulcular debridement, osteoplasty and osseous recontouring, and osseous crown lengthening.

FIGS. 1-3 and 5-7 illustrate embodiments of the present invention in which the system console 12 has two console connector assemblies 22, to one of which a handpiece assembly 14 is attached via a mating handpiece connector assembly 30. FIG. 4 illustrates and embodiment of the present invention in which four handpieces 58 are attached to the console 54 via respective mating console connector assemblies, attached to an exterior of the console housing 56, and handpiece connector assemblies 60. It will be appreciated that these embodiments are presented for illustration purpose only. The system of the present invention is adapted for performing at least one procedure of the group consisting of: treatment procedures and diagnostic procedures and is capable of including multiple handpiece assemblies 14, wherein the console 12 is capable of including one or multiple connectors for attaching the handpiece assemblies 14. The handpiece assemblies 14 are capable of being detachably connected with the console 12 via the handpiece connector assembly 30 and corresponding console connector assembly 20.

In one embodiment, the subject system for dental applications further comprises a handpiece kit (the kit not shown in the drawing). The handpiece kit includes multiple interchangeable shaped head modules 78 and multiple interchangeable tip modules 80, as illustrated by FIG. 5, FIG. 6, and FIG. 7. In one implementation of this embodiment, the distal part 28 of the handpiece assembly 14, is of a modular structure. In this embodiment, the distal part of the at least one handpiece assembly 14 includes a shaped head module 78 detachably connected with the tip module 80. The shaped head module 78 and the tip module 80 are selected from the handpiece kit in accordance with a requirement of a treatment procedure or diagnostic procedure.

The handpiece assembly 14 is preferably selected from the group consisting of: a handpiece assembly for laser cutting of soft or hard tissue, a handpiece assembly for dental whitening, a handpiece assembly for optical curing, and a handpiece assembly for optical imaging or diagnostics.

In one embodiment, the handpiece assembly 14 further comprises sensors for feedback control (not shown in the drawings), using acoustical, optical or electrical sensors. The handpiece assembly 14 is further capable of including an illumination system (not shown in the drawings).

The system for dental applications preferably includes a system kit (not shown in the drawings). The system kit includes multiple interchangeable handpiece assemblies 14. As will be appreciated by those skilled in the art, a necessary handpiece assembly 14 is selected from the system kit in accordance with a requirement of a treatment procedure or diagnostic procedure.

The present invention is not limited to the components shown in FIGS. 1-7. For example, for treatment of dental tissue water and high pressure air can be used for cooling tissue and other functions, such as cleaning of the treatment zone from products of ablation, control cutting and ablation process. Water and air or other liquid and gas can be delivered through the same connectors 20, 30 and umbilical 26 to the distal part 28 of handpiece assemblies 14. High pressure air can be delivered to the handpiece connector assembly 30 from high pressure air line in the dental chair. Valves to control air and liquid delivery can be located in the console 12 or in the handpiece assembly 14. High pressure gas can be used for scanning the laser beam position. The connector assemblies 20 and 30 in the console 12 and the proximal part 24 of the handpiece assembly 14, respectively, are adapted for mating with each other. A corresponding console and/or handpiece connector assembly 20, 30 may include one or several commercially available connectors or a special design for easy connection and disconnection. The connector assemblies 20, 30 22 may include electrical connectors for high electrical power for semiconductor light sources, electrical connectors for other sources, such as illumination lamp or LED, a scanner valve, electrical or optical connectors for command and signal transmission. In other embodiments signal transmission may be performed through a power cord and be wired or wireless. In addition, connector assemblies 20, 30 may include liquid and gas connectors. Those skilled in the art will recognize that data storage device of the computer 36 is capable of being implemented as internal storage component of the computer 36, such as, for example and without limitation, an internal hard disk drive, or the like.

In accordance with one embodiment of the subject invention, the data storage device of the computer 36 includes an operating system having a system registry, such as a WINDOWS-based operating system produced by the MICROSOFT CORPORATION or a UNIX-based operating system. In such an embodiment, the data storage device of the computer 36 includes one or more applications, as will be understood by those skilled in the art. Preferably, the data storage device of the computer 36 further includes means for receiving respective identification data calibration data, and operational data from the at least one handpiece assembly, means for receiving from the at least one handpiece assembly the at least one first executable code for a selected dental application associated with the adaptive console, means for storing corresponding identification data, calibration data, and operational data for the at least one handpiece assembly, means for storing the at least one first executable code for a selected dental application associated with the adaptive console received from the at least one handpiece assembly, means for identifying the at least one handpiece assembly by associating identification data received from the at least one handpiece assembly with corresponding identification data stored in the adaptive console first memory means, means for setting calibration and operational parameters of the adaptive console in accordance with received calibration and operational data, means for retrieving from the adaptive console second memory means the at least one first executable code received from the at least one handpiece assembly, and means for executing the at least one first executable code for a selected dental application received from the at least one handpiece assembly.

It will be appreciated that the data storage device of the microprocessor 76 is capable of being implemented as internal storage component of the microprocessor 76, such as, for example and without limitation, an internal hard disk drive, or the like. In accordance with one embodiment of the subject invention, the data storage device of the microprocessor 76 includes an operating system having a system registry, such as a WINDOWS-based operating system produced by the MICROSOFT CORPORATION or a UNIX-based operating system. In such an embodiment, the data storage device of the microprocessor 76 includes one or more applications, as will be understood by those skilled in the art. Preferably, the data storage device of the microprocessor 76 further includes means for storing respective identification data, calibration data, and operational data, means for outputting the respective identification data calibration data, and operational data, means for storing at least one first executable code for a selected dental application associated with the adaptive console, and output means for outputting the at least one first executable code for a selected dental applications associated with the adaptive console.

Calibration data may include conversion of laser energizing parameters (voltage, current, pulse duration etc) to the parameters of laser radiation (power, pulse energy, fluence etc). Calibration data may also include scanner sensitivity—conversion of applied voltage or current or air/liquid flow to scanning range or speed.

Operational data may include safe usage limits—pump voltage or current limitation, scanning limits, recommended procedures, recommended parameters to perform these procedures, and the like.

As will be recognized by those skilled in the art, a representative architecture of the computer 36 and/or the microprocessor 76 (not shown in the drawings), on which the above described operations of the subject system are completed, may suitably include a processor unit in data communication with read only memory, suitable non-volatile read only memory, volatile read only memory or a combination thereof, random access memory, display interface, and storage interface. Interface to the foregoing modules is capable of being accomplished via a bus, as known in the art.

The read only memory may include firmware, such as static data or fixed instructions, such as BIOS, system functions, configuration data, and other routines used for operation of the computer 36 and/or the microprocessor 76. The random access memory provides a storage area for data and instructions associated with applications and data handling accomplished by the computer 36 and/or the microprocessor 76.

The display interface receives data or instructions from other components on the bus, which data is specific to generating a display to facilitate a user interface. The display interface suitably provides output to a display terminal, suitably a video display device such as a monitor, LCD, plasma, touch screen, or any other suitable device, as known in the art. The user interface is also capable of including input means, such as touch screen, keyboard, mouse, joystick, trackball and other pointing devices known in the art. The user interface can also include more laser specific input means, such as foot pedal or switch.

The storage interface suitably provides a mechanism for non-volatile, bulk or long term storage of data or instructions in the computer 36 and/or the microprocessor 76. The storage interface suitably uses a storage mechanism, such as storage, suitably comprised of a disk, tape, CD, DVD, or other relatively higher capacity addressable or serial storage medium. The preferable storage includes electrically programmable random access or read only memory (EEPROM or EEPRAM), flash memory etc. The storage may be embedded in a microprocessor as many models have some non-volatile embedded storage, for example PIC series processors from Microchip Technology Inc. The storage also is capable of being embedded into specialized chips like 1-wire from Maxim semiconductors, or I²C-interface chips made by multiple vendors.

In operation, corresponding identification data, calibration data, and operational data for at least one handpiece assembly is stored in a storage associated with the adaptive console during manufacture of the adaptive console. Corresponding identification data, calibration data, and operational data for at least one handpiece assembly is stored in a storage associated with the at least one handpiece assembly during manufacture of the at least one handpiece assembly. At least one first executable code for a selected dental application associated with the adaptive console is stored in a storage associated with the at least one handpiece assembly during manufacture of the at least one handpiece assembly.

Upon attaching the at least one associated handpiece assembly to the adaptive console such that the at least one associated handpiece assembly is in communication with the adaptive console, the at least one associated handpiece assembly outputs data indicative of identification data, calibration data, and operational to the adaptive console. This data is received by the adaptive console, whereupon the adaptive console identifies the at least one associated handpiece assembly by associating data received from the at least one associated handpiece assembly with corresponding data stored in the storage associated with the adaptive console.

Once the at least one associated handpiece assembly is identified, a corresponding at least one first executable code for a selected dental application associated with the adaptive console is receiving from the at least one associated handpiece assembly and stored in an associated storage. A compatibility check is then performed of the at least one handpiece assembly with the adaptive console by associating software and hardware versions of the handpiece assembly with software and hardware versions of the adaptive console. The calibration and operational parameters of the adaptive console are then set in accordance with received calibration and operational data and the at least one first executable code for a selected dental application received from the at least one associated handpiece assembly is executed. At least one first handpiece specific user interface in accordance with the at least one first executed executable code for a selected dental application received from the at least one associated handpiece assembly is then displayed.

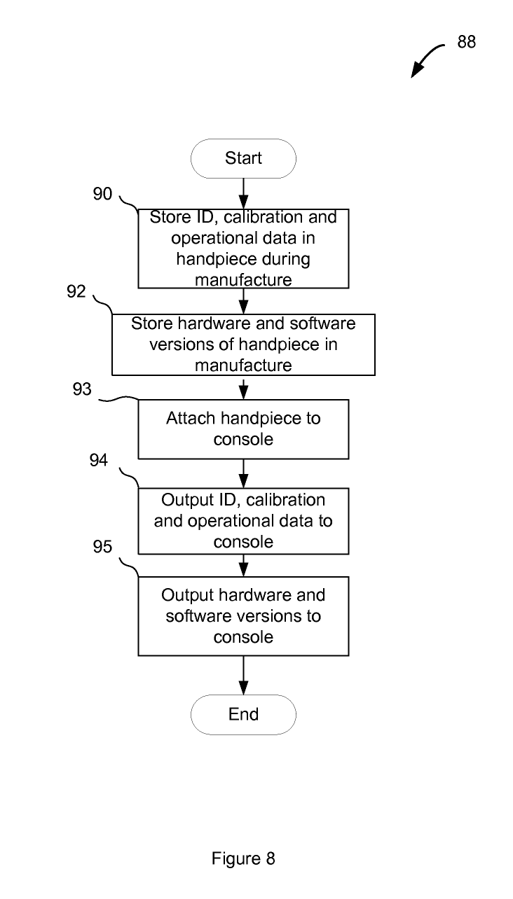

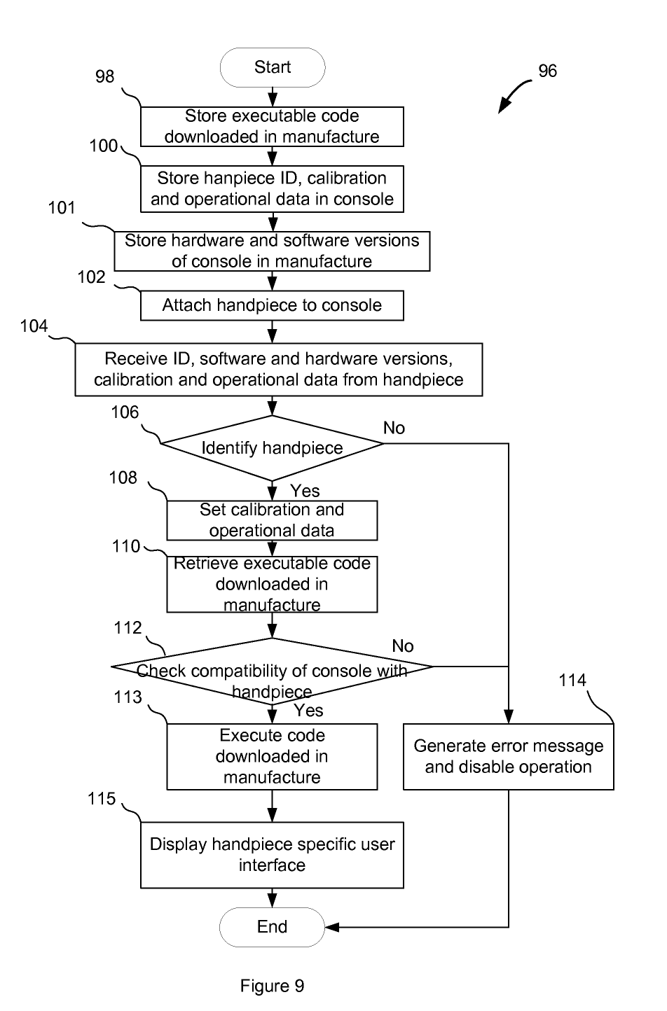

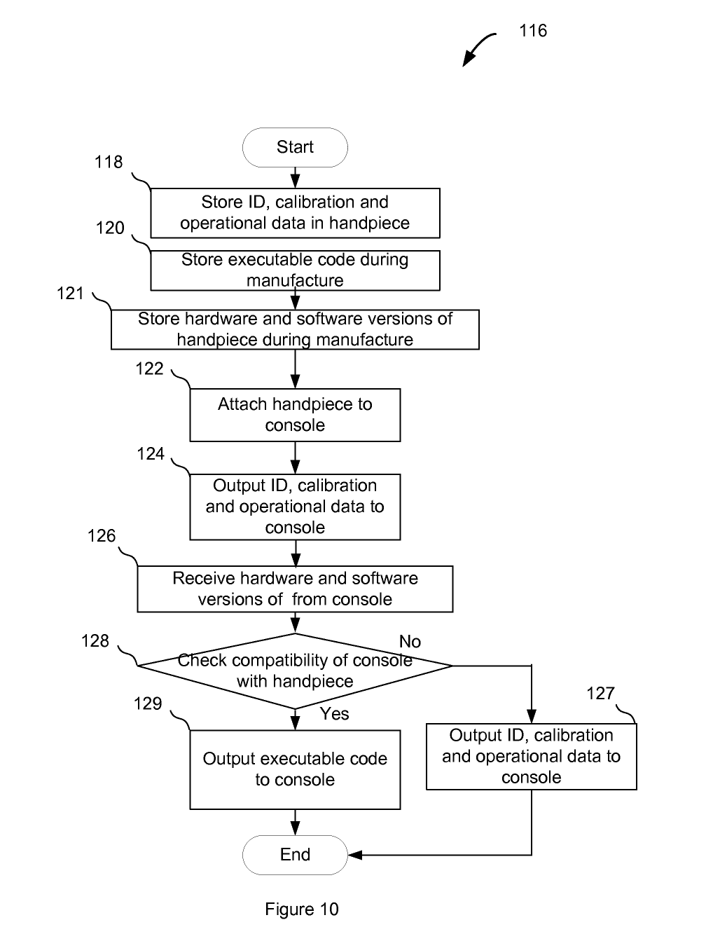

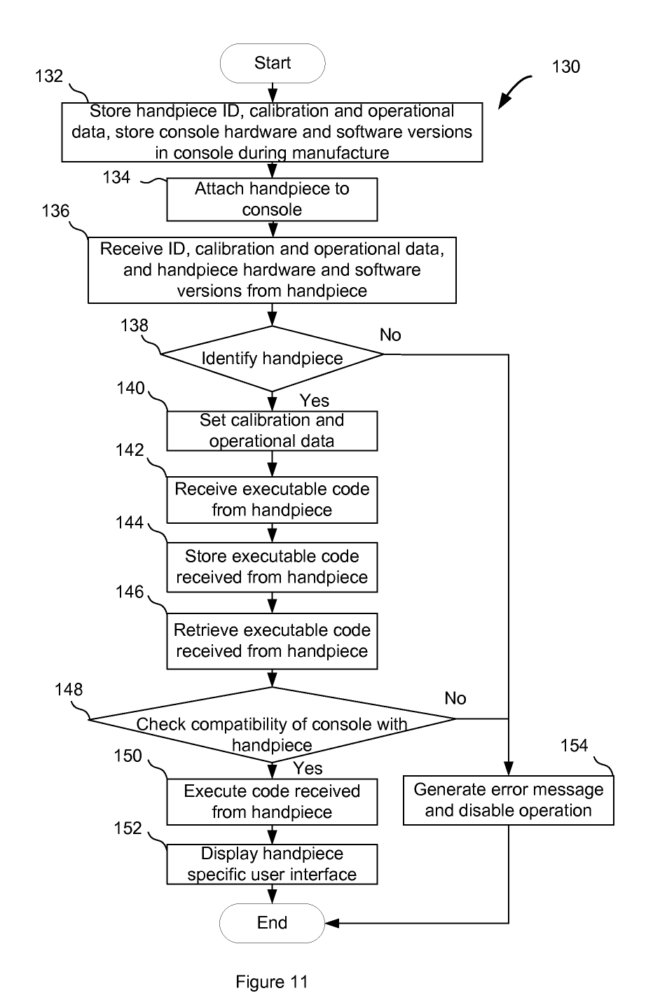

The functioning of the various embodiments of the subject system for dental applications described above in accordance with FIG. 1, FIG. 2, FIG. 3, FIG. 4, FIG. 5, FIG. 6, and FIG. 7 will better be understood in conjunction with the method illustrated in FIG. 8, FIG. 9, FIG. 10, and FIG. 11. Referring now to FIG. 7, there is shown a flowchart 88 illustrating the method in accordance with one embodiment of the subject invention. As shown in FIG. 8, the flowchart 88 illustrates the handpiece-side of operations of the method in accordance with the subject invention. Beginning at step 90, corresponding identification data, calibration data, and operational data for at least one handpiece assembly 14 is stored in a storage associated with the at least one handpiece assembly 14 during manufacture of the handpiece assembly 14. Flow then proceeds to step 92, at which step the hardware and software versions of handpiece assembly 14 are stored during manufacture of the handpiece assembly 14. Next, at step 93, the handpiece assembly 14 is attached to the console 12 via corresponding mating connector assemblies 30 and 20. Once the handpiece assembly 14 is attached to the console 12, the microprocessor 76 of the handpiece assembly 14, at step 94 outputs corresponding identification data, calibration data, and operational data to the console 12 and at step 95, the handpiece assembly outputs the hardware and software versions of handpiece assembly 14, which are stored during manufacture of the handpiece assembly 14. The method in accordance with this embodiment of the subject invention, then terminates from the handpiece-side.

Referring now to FIG. 9, there is shown a flowchart 96 illustrating the method in accordance with one embodiment of the subject invention, from the console-side. Beginning at step 98, at least one executable code for a selected dental application downloaded during manufacture of the adaptive console 12, is stored in a storage associated with the adaptive console 12. At step 100, corresponding identification data, calibration data, and operational data for at least one handpiece assembly 14 is stored in a storage associated with the adaptive console 12. At step 101 hardware and software versions of the adaptive console 12 are stored during manufacture of the console 12. Flow then proceeds to step 102, at which step the handpiece assembly 14 is attached to the adaptive console 12 via corresponding mating connector assemblies 30 and 20. Once the handpiece assembly 14 is attached to the console 12, the computer 36 of the adaptive console 12, at step 104 receives data indicative of identification data, calibration data, operational data, and data indicative of the hardware and software versions of the handpiece assembly 14, from the at least one associated handpiece assembly 14. Flow then proceeds to step 106, whereupon an identification process is performed by the computer 36 of the adaptive console 12. Upon a negative determination, that is when the computer 36 does not identify the handpiece assembly 14 based on received identification data, flow proceeds to step 114. At step 114 an error message is generated to disable the process, whereupon the process terminates. In the event of a positive determination, that is when the computer 36 does identify the handpiece assembly 14, flow proceeds to step 108.

At step 108, calibration and operational parameters of the adaptive console 12 are set in accordance with received calibration and operational data. Once calibration and operational parameters of the adaptive console 12 are set, flow proceeds to step 110, at which step the computer 36 retrieves the at least one executable code for a selected dental application downloaded during manufacture of the adaptive console 12, from an associated storage. Following retrieval, at step 112 a compatibility check is performed by the computer 36 of the console 12. The compatibility check is performed by associating software and hardware versions of the handpiece assembly 14 with software and hardware versions of the console 12. When the computer 36 determines at step 112, that software and hardware versions of the handpiece assembly 14 are not compatible with software and hardware versions of the console 12, flow proceeds to step 114. At step 114 an error message is generated to disable the process, whereupon the process terminates. Upon a positive determination at step 112, that is when it is determined that software and hardware versions of the handpiece assembly 14 are compatible with software and hardware versions of the console 12, the executable code for a selected dental application downloaded during manufacture of the adaptive console 12, is executed by the computer 36, at step 114. Following execution of the executable code, a handpiece specific user interface in accordance with executed executable code for a selected dental application, is displayed on the display 68, at step 115. As will be appreciated by those skilled in the art, the compatibility check at step 112 is capable of being performed before the step 108 of setting the calibration and operational parameters of the adaptive console 12.

Turning now to FIG. 10, there is shown a flowchart 116 illustrating the method in accordance with another embodiment of the subject invention. As shown in FIG. 10, the flowchart 116 illustrates the handpiece-side of operations of the method in accordance with the subject invention. Beginning at step 118, corresponding identification data, calibration data, and operational data for at least one handpiece assembly 14 is stored in a storage associated with the at least one handpiece assembly 14. Flow then proceeds to step 120, at which step, a corresponding executable code for a selected dental application associated with the adaptive console, is stored in a storage associated with the handpiece assembly 14 during manufacture of the handpiece assembly 14. At step 121, hardware and software versions are stored in a storage associated with the handpiece assembly 14 during manufacture of the handpiece assembly 14. Flow then proceeds to step 122. At step 122, the handpiece assembly 14 is attached to the console 12 via corresponding mating connector assemblies 30 and 20. Once the handpiece assembly 14 is attached to the console 12, the microprocessor 76 of the handpiece assembly 14, at step 124, outputs corresponding identification data, calibration data, and operational data to the console 12. Flow then proceeds to step 126, at which step the handpiece assembly 14 receives hardware and software versions of the adaptive console 12, whereupon a compatibility check is performed by the microprocessor 76 of the handpiece assembly 14. When the microprocessor 76 determines at step 128, that software and hardware versions of the handpiece assembly 14 are not compatible with software and hardware versions of the console 12, flow proceeds to step 127. At step 127 an error message is generated to disable the process, whereupon the process terminates. Upon a positive determination at step 128, that is when it is determined that software and hardware versions of the handpiece assembly 14 are compatible with software and hardware versions of the console 12, the executable code for a selected dental application stored in the handpiece assembly 14 is output to the console 12, at step 129. The method in accordance with this embodiment of the subject invention, then terminates from the handpiece-side.

Referring now to FIG. 11, there is shown a flowchart 130 illustrating the method in accordance with one embodiment of the subject invention, from the console-side. Beginning at step 132, corresponding identification data, calibration data, and operational data for at least one handpiece assembly 14, and console hardware and software versions are stored in a storage associated with the adaptive console 12. Flow then proceeds to step 134, at which step the handpiece assembly 14 is attached to the adaptive console 12 via corresponding mating connector assemblies 30 and 20. Once the handpiece assembly 14 is attached to the console 12, the computer 36 of the adaptive console 12, at step 136 receives data indicative of identification data, calibration data, and operational data, and data indicative of hardware and software versions of the handpiece assembly 14, from the at least one associated handpiece assembly 14. Flow then proceeds to step 138, whereupon an identification process is performed by the computer 36 of the adaptive console 12. Upon a negative determination, that is when the computer 36 does not identify the handpiece assembly 14 based on received identification data, flow proceeds to step 154. At step 154 an error message is generated to disable the process, whereupon the process terminates. In the event of a positive determination, that is when the computer 36 does identify the handpiece assembly 14, flow proceeds to step 140.

At step 140, calibration and operational parameters of the adaptive console 12 are set in accordance with received calibration and operational data. Once calibration and operational parameters of the adaptive console 12 are set, flow proceeds to step 142, at which step the computer 36 receives at least one executable code for a selected dental application, from the handpiece 14. Following receiving, at step 144, the executable code is stored in a storage associated with the console 12. At step 146, the executable code is retrieved from the associated storage and a compatibility check is initiated and performed by the computer 36 of the console 12, at step 148. The compatibility check is performed by associating software and hardware versions of the handpiece assembly 14 with software and hardware versions of the console 12. When the computer 36 determines at step 148, that software and hardware versions of the handpiece assembly 14 are not compatible with software and hardware versions of the console 12, flow proceeds to step 154. At step 154 an error message is generated to disable the process, whereupon the process terminates. Upon a positive determination at step 148, that is when it is determined that software and hardware versions of the handpiece assembly 14 are compatible with software and hardware versions of the console 12, the executable code for a selected dental application received from the handpiece assembly 14, flow proceeds to step 150. At step 150, the executable code received from the handpiece assembly 14, is executed by the computer 36. Following execution of the executable code, a handpiece specific user interface in accordance with executed executable code for a selected dental application, is displayed on the display 68, at step 152.

The foregoing descriptions of the preferred embodiments of the subject invention have been presented for purposes of illustration and description. They are not intended to be exhaustive or to limit the subject invention to the precise form disclosed. Obvious modifications or variations are possible in light of the above teachings. The embodiments were chosen and described to provide the best illustration of the principles of the subject invention and its practical application to thereby enable one of ordinary skill in the art to use the subject invention in various embodiments and with various modifications as are suited to the particular use contemplated. All such modifications and variations are within the scope of the subject invention as determined by the appended claims when interpreted in accordance with the breadth to which they are fairly, legally and equitably entitled. The use of “such as” and “for example” are only for the purposes of illustration and do not limit the nature or items within the classification.

### INDUSTRIAL APPLICABILITY

The present invention is capable of being implemented for various dental applications, such as surgical, microsurgical, cosmetic, and diagnostics procedures, for example, for diagnostics of conditions of individual organs and systems of the oral cavity and adjacent tissues, including, in vivo examinations, and for technical diagnostics, for example, for monitoring technological processes. The invention is capable of being implemented using standard components and elements, as known in the art.

1. A system for dental applications comprising:
a console comprising:
a console housing;
electrical energizing means placed inside the console housing;
at least one console connector assembly attached to an exterior of the console housing, the at least one console connector assembly comprising a console electrical connector unit; and
user interface means; and
at least one handpiece assembly comprising:
a proximal part including a handpiece connector assembly adapted for mating with a corresponding console connector assembly, the handpiece connector assembly comprising a handpiece electrical connector unit adapted for mating with a corresponding console electrical connector;
an umbilical comprising an optical fiber or electrical wires or both; and
a distal part for optical interaction with hard or soft tissue, the distal part of the at least one handpiece assembly being in communication with the proximal part of the at least one handpiece assembly via the umbilical;
wherein the system for dental applications is adapted for performing treatment procedures or diagnostic procedures or both.

2. (canceled)

3. The system for dental applications of claim 1, wherein the console further comprises electronic processing means placed inside the console housing.

4. (canceled)

5. The system for dental applications of claim 1, wherein the at least one handpiece assembly is detachably connected with the console via the handpiece connector assembly and corresponding console connector assembly.

6. The system for dental applications of claim 1, wherein the at least one handpiece assembly further comprises a semiconductor converter of electrical power to optical power.

7. The system for dental applications of claim 6, wherein the semiconductor converter of electrical power to optical power is a diode laser and is placed in the handpiece connector assembly, and wherein the umbilical comprises fiber for delivering light to the distal part of the handpiece assembly.

8-13. (canceled)

14. The system for dental applications of claim 1, wherein the distal part of the at least one handpiece assembly is detachably connected with the umbilical.

15. The system for dental applications of claim 14, further comprising a handpiece kit, wherein the handpiece kit comprises multiple interchangeable shaped head modules and multiple interchangeable tip modules.

16. (canceled)

17. The system for dental applications of claim 1, wherein the at least one handpiece assembly is selected from the group consisting of: a handpiece assembly for laser cutting of soft or hard tissue, a handpiece assembly for dental whitening, a handpiece assembly for optical curing, and a handpiece assembly for optical imaging or diagnostics.

18. The system for dental applications of claim 1, wherein the at least one handpiece assembly further assembly comprises sensors for feedback control.

19-21. (canceled)

22. The system for dental applications of claim 1, further comprising a system kit, the system kit comprising multiple interchangeable handpiece assemblies, wherein the at least one handpiece assembly is selected from the system kit in accordance with a requirement of a treatment procedure or diagnostic procedure.

23. The system for dental applications of claim 1:
wherein the at least one handpiece assembly further comprises:
handpiece first memory means for storing respective identification data, calibration data, and operational data; and
handpiece first output means for outputting the respective identification data calibration data, and operational data;
wherein the console further comprises:
first receiving means for receiving respective identification data calibration data, and operational data from the at least one handpiece assembly;
console first memory means for storing at least one executable code for a selected dental application, the at least one executable code being downloaded during manufacture of the console;
console second memory means for storing corresponding identification data, calibration data, and operational data for the at least one handpiece assembly;
identifying means for identifying the at least one handpiece assembly by associating identification data received from the at least one handpiece assembly with corresponding identification data stored in the console second memory means;
retrieving means for retrieving the at least one executable code for a selected dental applications from the console first memory means;
setting means for setting calibration and operational parameters of the console in accordance with received calibration and operational data; and
management means for executing the at least one executable code downloaded during manufacture of the console; and
wherein the user interface means is adapted for displaying at least one handpiece specific user interface in accordance with the at least one executed executable code for a selected dental application downloaded during manufacture of the console.

24. The system for dental applications of claim 23, wherein at least one of the at least one handpiece assembly or the console further comprises additional memory means for storing a compatibility matrix and compatibility checking means for checking compatibility of the at least one handpiece assembly with the console by associating software and hardware versions of the handpiece assembly with software and hardware versions of the console.

25. (canceled)

26. The system for dental applications of claim 1:
wherein the console is made adaptive;
wherein the at least one handpiece assembly further comprises:
handpiece first memory means for storing respective identification data, calibration data, and operational data; and
handpiece first output means for outputting the respective identification data calibration data, and operational data;
handpiece second memory means for storing at least one first executable code for a selected dental application associated with the adaptive console; and
handpiece second output means for outputting the at least one first executable code for a selected dental applications associated with the adaptive console;
wherein the adaptive console further comprises:
first receiving means for receiving respective identification data calibration data, and operational data from the at least one handpiece assembly;
second receiving means for receiving from the at least one handpiece assembly the at least one first executable code for a selected dental application associated with the adaptive console;
adaptive console first memory means for storing corresponding identification data, calibration data, and operational data for the at least one handpiece assembly;
adaptive console second memory means for storing the at least one first executable code for a selected dental application associated with the adaptive console received from the at least one handpiece assembly;
identifying means for identifying the at least one handpiece assembly by associating identification data received from the at least one handpiece assembly with corresponding identification data stored in the adaptive console first memory means;
setting means for setting calibration and operational parameters of the adaptive console in accordance with received calibration and operational data;
first retrieving means for retrieving from the adaptive console second memory means the at least one first executable code received from the at least one handpiece assembly; and
first management means for executing the at least one first executable code for a selected dental application received from the at least one handpiece assembly;
wherein the user interface means is adapted for displaying at least one first handpiece specific user interface in accordance with the at least one first executed executable code for a selected dental application received from the at least one handpiece assembly.

27-29. (canceled)

30. The system for dental applications of claim 26, wherein if multiple first executable codes are stored in handpiece second memory means, the multiple first executable codes belong to different platforms.

31. The system for dental applications of claim 1, wherein the user interface means includes a graphical display.

32. The system for dental applications of claim 1, wherein the at least one procedure is selected from procedures for oral cavity treatment, evaluation, prevention or diagnose conditions in the oral cavity or the jaw with light sources suitable for drilling, or cutting hard tissue, soft tissue, or dental material, or irreversible modification by phase transition of the structure of hard tissue.

33. The system for dental applications of claim 1, wherein the at least one procedure is selected from the group of diagnostic procedures consisting of: optical imaging, translucent imaging, fluorescent imaging, fluorescent spectroscopy, fluorescent imaging, confocal microscopy, multiphoton microscopy, reflectometry and optical coherence tomography.

34-36. (canceled)

37. A system for dental applications comprising:
an adaptive console comprising:
a console housing;
electrical energizing means placed inside the console housing; and
at least one console connector assembly attached to an exterior of the console housing; and
at least one handpiece assembly for optical interaction with hard or soft tissue comprising:
a handpiece connector assembly adapted for mating with a corresponding console connector assembly;
handpiece first memory means for storing respective identification data, calibration data, and operational data; and
handpiece first output means for outputting the respective identification data calibration data, and operational data;
handpiece second memory means for storing at least one first executable code for a selected dental application associated with the adaptive console; and
handpiece second output means for outputting the at least one first executable code for a selected dental applications associated with the adaptive console;
wherein the console further comprises:
first receiving means for receiving respective identification data calibration data, and operational data from the at least one handpiece assembly;
second receiving means for receiving from the at least one handpiece assembly the at least one first executable code for a selected dental application associated with the adaptive console;
adaptive console first memory means for storing corresponding identification data, calibration data, and operational data for the at least one handpiece assembly;
adaptive console second memory means for storing the at least one first executable code for a selected dental application associated with the adaptive console received from the at least one handpiece assembly;
identifying means for identifying the at least one handpiece assembly by associating identification data received from the at least one handpiece assembly with corresponding identification data stored in the adaptive console first memory means;
setting means for setting calibration and operational parameters of the adaptive console in accordance with received calibration and operational data;
first retrieving means for retrieving from the adaptive console second memory means the at least one first executable code received from the at least one handpiece assembly;
first management means for executing the at least one first executable code for a selected dental application received from the at least one handpiece assembly; and
user interface means adapted for displaying at least one first handpiece specific user interface in accordance with the at least one first executed executable code for a selected dental application received from the at least one handpiece assembly; and
wherein the system for dental applications is adapted for performing treatment procedures or diagnostic procedures or both.

38. The system for dental applications of claim 37, wherein at least one of the at least one handpiece assembly or the console further comprises additional memory means for storing a compatibility matrix and compatibility checking means for checking compatibility of the at least one handpiece assembly with the console by associating software and hardware versions of the handpiece assembly with software and hardware versions of the console.

39-41. (canceled)

42. A handpiece assembly for dental applications comprising:
a proximal part including a handpiece connector assembly adapted for mating with a corresponding console connector assembly of an associated console;
an umbilical comprising an optical fiber or electrical wires or both; and
a distal part for optical interaction with hard or soft tissue, or dental material, the distal part of the handpiece assembly being in communication with the proximal part of the handpiece assembly via the umbilical;
wherein the handpiece assembly is adapted for performing treatment procedures or diagnostic procedures or both.

43. (canceled)

44. The handpiece assembly for dental applications of claim 42, wherein the handpiece assembly further comprises a semiconductor converter of electrical power to optical power.

45. The handpiece assembly for dental applications of claim 44, wherein the semiconductor converter of electrical power to optical power is a diode laser and is placed in the handpiece connector assembly, and wherein the umbilical comprises fiber for delivering light to the distal part of the handpiece assembly.

46-63. (canceled)
